(19) United States
(12) Patent Application Publication
(10) Pub. No.: US 2024/0215626 A1
(43) Pub. Date: Jul. 4, 2024

(54) A DRY FOOD PRODUCT COMPRISING FUNGAL BIOMASS AND METHODS FOR MANUFACTURING A DRIED FUNGAL BIOMASS FOOD PRODUCT

(71) Applicant: MYCORENA AB, Göteborg (SE)

(72) Inventors: Anton Johansson, Göteborg (SE); Ramkumar Nair, Västra Frölunda (SE); Ebba Fröling, Göteborg (SE); Frida Persson, Borås (SE); Paulo Gonçalves Teixeira, Göteborg (SE); Thibaut Riedel, Avignon (FR)

(21) Appl. No.: 18/556,903

(22) PCT Filed: Apr. 26, 2022
(86) PCT No.: PCT/EP2022/061083

§ 371 Date: Oct. 24, 2023

(30) Foreign Application Priority Data
Apr. 27, 2021 (SE) 2150532-6

(51) Int. Cl. A23L 31/00; A23J 1/00; A23J 3/20; A23L 3/44; A23L 29/212; A23L 29/30; A23L 33/115; A23L 33/185; A23L 33/195

(52) U.S. Cl. CPC A23L 31/00; A23J 1/008; A23J 3/20; A23L 3/44; A23L 29/212; A23L 29/30; A23L 33/115; A23L 33/185; A23L 33/195

(57) ABSTRACT

Described is a dry food product that includes fungal biomass, the fungal biomass has a filamentous mycelium network and wherein the dry food product has a water content within the range of from 0 to 10 wt. %. Also described is a method of manufacturing a dry fungal biomass food product.

### TECHNICAL FIELD

The present disclosure pertains to a rehydratable dry food product or ingredient comprising of fungi mycelium biomass. This disclosure also relates to methods for dehydrating food products to obtain products such as the one described, and the use of said food product either as a component in a final consumer product, or as a manufacturer ingredient.

### BACKGROUND

Dry foods have been used worldwide for widely different reasons. The main reason for the use of dry foods is however its extensive shelf life due to the low water activity, derived from the low water content of these. This extended shelf life, together with low weight due to the absence of water, makes dry foods extremely useful for outdoors applications such as camping, sports, but also as ration foods in military settings, rescue operations, and long-duration travels (including arctic and sea expeditions, as well as space exploration). Dry foods can be divided into products that are consumed in their dry state, and ones that are intended to be rehydrated before consumption. Rehydrated products have also in the recent decades become convenience foods, in which meals can be easily prepared by just adding water to the dry product for a fully reconstituted meal.

Nutritional protein in rehydratable foods can have both animal and plant-based origins. The use of freeze dry meat is a possibility in these meals and presents a nutritious protein source. The main disadvantages of freeze dry meat are the high costs of production from the freeze drying process, but also the brittleness of the dry meat, which tends to be diminished to powder from impact over transportation cycles. Moreover, with a growing shift to vegan diets for environmental, ethical or health reasons, the largest trend of protein sources in rehydratable meals come either from the use of dry raw vegetables, or from dry texturized vegetable protein (TVP), often originated from soy protein. TVP is the dry ingredient more commonly used as a meat replacement for vegan rehydratable dishes, as well as used by food manufacturers as a mince replacement in the production of fresh or frozen final consumer products. TVP is, however, limited since its texture is often not meat-like, it lacks the possibility to include flavours inside the dry material and lacks a good rehydratability when used in large pieces.

Mycoprotein and fungi-based meat replacements are a rising trend to build products that are direct replacements to meat, soy- and other plant-based products. However, in the current market mycoprotein is only supplied as fresh or frozen, whether this is as a final product or an ingredient. Production and use of dry mycoprotein products that can preserve its texture and taste when rehydrated would enable new applications, ease the use of mycoprotein in industry, and also allow for more economical and environmentally sustainable distribution processes.

A key aspect to rehydratable products is its capacity to uptake water in the same amount as its equivalent in a fresh form and do so in a way that it reconstitutes to its original form, structure, and consequently, texture. As such, for a successful rehydratable mycoprotein product, a high water absorption capacity, ability to keep the fungi mycelium structure, and ability to keep colour, are essential.

Dehydration methods in food industry for solid materials mostly involve oven or hot air drying through convection at temperatures between 60-120° C., vacuum-aided drying at temperatures between 45-60° C. and vacuum values of 30-80 mbar, and freeze drying which is performed on frozen material at −5° C. to −20° C. and vacuum values below 1 mbar. Freeze drying is a technique that creates sublimation of ice crystals in a frozen product, and often creates a porous material with better properties for rehydration. Freeze drying is however a lengthy, energy-consuming and consequently very costly process, and its effects on a fungal mycelial structure or mycoprotein product seem so far unknown. The causes for the high costs of this process are related to lengthy operation times, often between 48 h and 72 h, energy spent on freezing the product, and the energy to create very low vacuum levels. The process is therefore often not economically viable to be used in most common consumer products due to the high resulting price.

Thus, there is an increasing need to deliver high quality rehydratable products based on fungi mycelium which are highly rehydratable in a few minutes, and are reconstituted to its full characteristics of texture, shape and colour when rehydrated as final products or as an ingredient. Furthermore, there is a need for a cost-efficient process to produce said rehydratable fungi mycelium-based products that delivers low cost and low processing time.

### SUMMARY

One or more of the above objects may be achieved with a dry food product in accordance with claim 1, the use of the food product according to claim 18 and methods of manufacturing a dried fungal biomass food product according to claims 19, 24 and 27. The dry food product may also be denoted a rehydratable food product.

A dry food product as disclosed herein comprises fungal biomass. The fungal biomass comprises a filamentous mycelium network and the dry food product has a water content within the range of from 0 wt to 18 wt %.

Consumption of mycoprotein is associated with a range of benefits to health and wellbeing. The dry food product comprising fungal biomass according to the present disclosure provides a food product with prolonged shelf life and/or which is easily rehydratable and/or which reconstitutes to its original form, structure and consequently, texture after being rehydrated.

The fungal biomass comprises food-safe fungi, such as food safe filamentous fungi of the Zygomycota and/or Ascomycota phylum, excluding yeasts, such as fungi of the genera Rhizopus, Neurospora, Aspergillus, Trichoderma, Pleurotus, Ganoderma, Inonotus, Cordyceps, Ustilago, Tuber, Fusarium, Pennicillium, Xylaria, Trametes, or any combination thereof. Examples of food safe filamentous fungal species include, but are not limited to, Aspergillus oryzae, Rhizopus oryzae, Rhizopus oligosporus and Rhizopus microsporus, Fusarum graminareum, Cordyceps militars, Cordyceps sinensis, Tuber melanosporum, Tuber magnatum, Pennicillium camemberti, Neurospora intermedia, Neurospora sitophila, Xylaria hypoxion or any combination thereof.

The dry food product may have a water content within the range of from 0 to 10 wt %, optionally within the range of from 0 to 8 wt %, such as 0 to 5 wt, or within the range of from 0 to 2.5 wt %.

Optionally, 50% or more, such as 70% or more, or 80% or more of the filamentous mycelium network are aligned substantially in planes extending in a first direction, thus forming a lamellar structure.

It has surprisingly been found by the present inventor that when 50% or more, such as 70% or more, or 80% or more of the filamentous mycelium network are aligned substantially in planes, thus forming a lamellar structure, in a dry food product according to the present invention, the food product becomes easily rehydratable and reconstitutes to its original form, structure and consequently, texture after being rehydrated. The alignment of the filamentous mycelium network may thus provide a dry food product which, optionally after being reconstituted with water or another liquid, has an appealing texture and/or mouthfeel.

Said lamellar structure may be generated using any device that applies a unidirectional pressure on the mycelium network at an open surface, which allows the fibers to expand longitudinally during pressing and thus generating substantially aligned fibers. Such devices may be any device that creates a unidirectional pressing with mechanical force such as an hydropress, or alternatively devices that exert this force through pneumatics, hydraulics or mechanical wheels.

The protein content of the fungal biomass may be at least 50% per dry weight, such as from 50 to 75% per dry weight, based on the dry weight of the fungal biomass.

The dry food product may have a water activity of 0.8 or lower, optionally 0.6 or lower. A low water activity may be associated with a lower microbial growth and therefore extended shelf-life.

The dry fungal biomass food product may be a controlled low-temperature vacuum dehydrated food product. Such a low-temperature vacuum dehydrated food product is typically kept at temperatures within the range of from 0° C. and 15° C., here termed “chilled vacuum dehydration”. It has been found by the present inventors that chilled vacuum dehydration is a beneficial drying method for a food product comprising or consisting of fungal biomass. The fungal biomass food product may be dried in a cost-efficient process and prepare a high quality rehydratable products based on fungi mycelium being highly rehydratable in a few minutes, and are reconstituted to its full characteristics of texture, shape and colour when rehydrated as final products or as an ingredient.

The dry fungal biomass food product may alternatively be a freeze-dried food product, such as a freeze dried consumer end product.

The dry food product may have a water absorption capacity (WAC) within the range of from 70% to 200%, optionally within the range of from 80% to 180%, optionally within the range of from 90% to 180%, according to the water absorption capacity (WAC) test as disclosed herein. The fact that the dried food product has a high water absorption capacity, such as being within the range of from 70% to 200% of the total weight of the dry food product provide an improved food product after rehydration of the dry food product, such as rehydration performed by the end consumer or by the manufacturer when the dry food product is being provided as a manufacturer ingredient.

The dry food product may have a maximum rehydration rate of from 0 to 15 minutes, preferably 0 to 3 minutes, such as 0.1 to 3 minutes. The maximum rehydration rate is calculated by soaking between 1 and 10 g of the dry food product completely in water at 20° C., the weight of the product being measured with an interval of 1 to 5 minutes by taking the product sample out from the water and placing it on a scale with an accuracy of 0.01 g until the weight is constant.

A food additive may be present in the dry food product comprising fungal biomass in an amount of 0.05% by weight or more based on the total weight of the fungal biomass (including any water) and the food additive, i.e. of the total weight of the dry food product, wherein the food additive is integrated in (blended into) the filamentous mycelium network. “Integrated in” in this context means that if a solid piece of the dry food product is taken, the additives are stuck inside the network of fungal fibers/hyphae. It has been found that dry food products comprising fungal biomass with food additive is integrated in the filamentous mycelium network provides benefits in terms of texture and taste of a meat- or fish-replacement product, including fillets or steak replacements after rehydration of the dry food product. The integrated food additive may be present in an amount of 0.05% by weight or more of based on the total weight of dry food product, optionally 0.1% by weight or more, optionally 0.5% by weight or more, or 2% by weight or more based on the total weight of dry food product. Optionally, integrated food additive is present in an amount within the range of from 0.05% to 40% by weight based on the total weight of the dry food product. Optionally, the integrated food additive is present in an amount within the range of from 0.01% to 20%, such as from 0.01% to 15%, such as from 0.5 to 12.5%, such as from 2% to 10% by weight based on the total weight the dry food product.

The filamentous mycelium network comprising the integrated food additive may be substantially intact, which means that the mycelium network itself has a connected structure. This provides an enhanced texture of the food product comprising fungal biomass after rehydration of the dry food product.

The integrated food additive may be selected from the group consisting of food fibers, starches, proteins, fats, oils, food flours, hydrocolloids, and gelling agents.

The integrated food additive may be selected from the group consisting of rice starch, potato starch, corn starch as well as other modified starches, potato fibers, bamboo fibers, pea fibers, oat fibers, canola oil, pea protein, soy protein, hydrocolloids such as methylcellulose, carrageenan and alginate, flavours and/or spices.

The dry food product comprising fungal biomass may comprise or consist of fungal biomass and optionally an additive and/or optionally water. For example, the dry food product comprising fungal biomass may comprise or consist of fungal biomass in an amount of from about 60 wt % to about 100 wt %, water in an amount of from about 0 to about 18 wt % and one or more additives in a total amount of from about 0 wt % to about 20 wt %.

The amount of fungal biomass in the dry food product is typically from about 60 wt % to about 100 wt %, such as from about 65 wt % to about 100 wt %, from about 70 wt % to about 100 wt %, from about 75 wt % to about 100 wt %, from about 80 wt % to about 100 wt %, from about 82 wt % to about 100 wt %, from about 85 wt % to about 100 wt %, from about 90 wt % to about 100 wt %, from about 92 wt % to about 100 wt %, or from about 95 wt % to about 100 wt % based on the total weight of the dry food product.

The food product may be packed in a water-impermeable package. The food product may be a rehydratable ready-to-eat product packed in a water-impermeable package, such as a paperboard laminate package provided with a polymeric inner layer and/or a metallic foil layer.

The dry food product may be an instant/rehydratable food product. For example, the dry food product may be a meat-replacement product, such as a chicken, pork, beef, lamb, or seafood replacement product, noodles, or a powder product.

The food product may be a dehydrated and rehydratable mince-like intermediate or final food product, such as dehydrated mince-like pieces, used to create a burger patty, meatballs, sausages, etc.

It has been found by the present inventors that high quality rehydratable products based on fungi mycelium which are highly rehydratable in a few minutes, and are reconstituted to its full characteristics of texture, shape and colour when rehydrated as final products or as an ingredient.

Accordingly, there is the need for a cost-efficient process to produce said rehydratable fungi mycelium-based products that delivers low cost and lower process time.

The present disclosure thus furthermore relates to a method for manufacturing a dried (i.e. dry) fungal biomass food product, the method comprising the steps of:
a) providing a solution or suspension comprising a fungal biomass, such as a fungal biomass comprising food-safe filamentous fungi as defined herein;
b) dewatering the fungal biomass to substantially orient the fungal biomass in a filamentous mycelium network in planes, for example such that 50% or more, such as 70% or more, or 80% or more of the filamentous mycelium network are aligned substantially in planes extending in a first direction, thus forming a lamellar structure, said dewatering being preferably made by applying a unidirectional pressing force to the fungal biomass, optionally such that a fungal biomass food product is obtained having a water content within the range of from 50 to 80% by weight, such as measured by weighing of the fungal biomass before and after an oven drying step; and
c) submitting the dewatered fungal biomass to chilled vacuum dehydration and/or freeze-drying, thereby obtaining a dried fungal biomass food product.

The fungal biomass may be heat treated before or after step a) in the above method, such as by heating the fungal biomass to a temperature within the range of from 50 to 95° C. The heating may e.g. be performed for a shorter time period if a temperature in the higher end of the range is used, and a longer time period if a temperature in the lower end of the range is used. For example, the fungal biomass may be heated for about 1.5 min at about 95° C. or about 20 min at about 65° C. Also, a combination of a higher and a lower temperature (in any order) may be used for heat treating the fungal biomass. The method may further comprises adding a food additive to the fungal biomass before step b) in the above method and mixing the fungal biomass and the food additive, thereby integrating the food additive into the filamentous mycelium network.

Step c) in the above method may comprise the steps of:
i) cooling the fungal biomass obtained in step b) to a temperature within the range of from 0° C. to 17° C.;
ii) maintaining the fungal biomass at a temperature within the range from 0° C. to 17° C. and subjecting the cooled fungal biomass to a vacuum pressure within the range of from 0.001 to 50 mbar, such as from 1 to 50 mbar, such as from 2 mbar to 50 mbar, or from 1 to 15 mbar, until the water content is 10% by weight or lower, optionally 8% by weight or lower, thereby obtaining a dried fungal biomass food product.

Steps i) and ii) may also be combined and performed simultaneously.

Step c) in the above method may alternatively comprise the steps of:
i) freeze-drying the fungal biomass to a temperature within the range of from −5° C. to −35° C.;
ii) maintaining the fungal biomass in a temperature within the range from −5° C. to −35° C. and subjecting the freeze-dried fungal biomass from step e) to a vacuum pressure within the range of from 0.001 mbar to 6 mbar until the water content is 10% by weight or lower, optionally 8% by weight or lower, thereby obtaining a freeze-dried fungal biomass food product.

The present disclosure furthermore relates to a method for manufacturing a dried fungal biomass food product, the method comprising the steps of:
a) providing a fungal biomass, such as a fungal biomass comprising food-safe filamentous fungi, such as by cultivating fungi under aerobic submerged fermentation conditions using a closed fermentation vessel with liquid substrate media while stirring to obtain a fungal biomass;
b) processing the fungal biomass obtained from step a) by heating to a temperature within the range of from 50 to 95° C. or 60-85° C.;
c) optionally separating the fungal biomass obtained from step b) from the liquid cultivation media, such as by filtration or centrifugation, optionally such as the biomass has a water content within the range of from 80 wt % to 98 wt %, this step may alternatively be performed before step b);
d) dewatering, such as by pressing or centrifuging, the fungal biomass obtained from step c) to substantially orient the fungal biomass in a filamentous mycelium network in planes, for example such that 50% or more, such as 70% or more, or 80% or more of the filamentous mycelium network are aligned substantially in planes extending in a first direction, thus forming a lamellar structure, optionally such that a fungal biomass food product is obtained having a water content within the range of from 50 to 80% by weight, such as measured by weighing of the fungal biomass before and after an oven drying step;
e) cooling the fungal biomass obtained in step d) to a temperature within the range of from 0° C. to 17° C., such as to a temperature within the range of from 0° C. to 15° C. or 0° C. to 12° C.;
f) maintaining the fungal biomass at a temperature within the range from 0° C. to 17° C., such as within the range from 0° C. to 15° C. or 0° C. to 12° C. and subjecting the cooled fungal biomass from step e) to a vacuum pressure within the range of from 0.001 to 50 mbar, such as from 1 to 50 mbar, such as from 2 mbar to 50 mbar, such as from 1 to 15 mbar, such as from 4 to 50 mbar, such as from 4 to 6 mbar until the water content is 10% by weight or lower, optionally 8% by weight or lower, thereby obtaining a dried fungal biomass food product,
g) optionally preparing a chilled vacuum dehydrated food product obtained from the chilled vacuum dehydrated fungal biomass in step f).

Steps e) and f) may be combined and performed simultaneously.

The present disclosure furthermore relates to a method for manufacturing a dried fungal biomass food product, the method comprising the steps of:
a) providing a fungal biomass, such as a fungal biomass comprising food-safe filamentous fungi, such as by cultivating fungi under aerobic submerged fermentation conditions using a closed fermentation vessel with liquid substrate media while stirring to obtain a fungal biomass;
b) processing the fungal biomass obtained from step a) by heating to a temperature within the range of from 50 to 95° C. or 60-85° C.;
c) optionally separating the fungal biomass obtained from step b) from the liquid cultivation media, such as by filtration or centrifugation, optionally such that the biomass has a water content within the range of from 80 wt % to 98 wt %, this step may alternatively be performed before step b);
d) dewatering, such as by pressing or centrifuging, the fungal biomass obtained from step c) to substantially orient the fungal biomass in a filamentous mycelium network in planes, for example such that 50% or more, such as 70% or more, or 80% or more of the filamentous mycelium network are aligned substantially in plane(s) extending in a first direction, thus forming a lamellar structure, optionally such that a fungal biomass food product is obtained having a water content within the range of from 50 to 80% by weight, such as measured by weighing of the fungal biomass before and after an oven drying step;
e) freeze-drying the fungal biomass to a temperature within the range of from −5° C. to −35° C., such as −5° C. to −35° C.;
f) maintaining the fungal biomass in a temperature within the range from −5° C. to −35° C., such as −5° C. to −35° C., and subjecting the freeze-dried fungal biomass from step e) to a vacuum pressure within the range of from 0.001 mbar to 6 mbar until the water content is 10% by weight or lower, optionally 8% by weight or lower, thereby obtaining a (freeze-) dried fungal biomass food product.

The dewatering step, step b) or d) in the above methods, may include dewatering the fungal biomass by pressing the fungal biomass with a force applied in a single direction, for example with a pressure of from 1.0 to 3.0 bar when using a hydropress. By pressing the fungal biomass, the fungal cells in the fungal biomass form a filamentous mycelium network. A pressing device for generating said unidirectional force in a single direction may be selected from hydropress, belt press and/or a batch-mode centrifugation device and the like. The water content of the fungal biomass before the dewatering step is preferably within the range of from about 80 wt % to about 98 wt %. This may e.g. be effected by separating the fungal biomass from a washing liquid or a liquid cultivation medium, such as by filtration or centrifugation.

The water content of the fungal biomass may be measured by weighing a sample of fungal biomass before and after an oven drying step or another drying method.

The heat treatment step is a process used for inactivating the fungal biomass after fermentation, whereby the fungal biomass together with the liquid fermentation substrate, a diluted version of it, or the biomass submerged in water, may be heated up to a temperature within the range of 50° to 95° C. or 60-85° C. e.g. for a period of time between 1.5 min and 1 h. The biomass may furthermore be heat treated before or after dewatering step (step b) or d in the above methods). The heat treatment may e.g. be effected by exposure to steam for 5-20 minutes at a temperature range of 50° to 95° C.

The fungal biomass may be washed before or after heat treatment, the fungal biomass may e.g. be rinsed with tap water, distilled water or water containing 0-5% of salt (NaCl), with a water temperature of 6-15° C., for 3-60 min.

Step f) may be performed in a vacuum chamber. The vacuum chamber may be connected to a condenser, optionally the condenser having a temperature within the range of from −50° C. to −90° C.

A food additive (as described elsewhere herein) may be added to the fungal biomass obtained before the dewatering step, e.g. in the step of separating the fungal biomass form the cultivation medium or washing liquid and mixed into the fungal biomass, thereby integrating the food additive into the filamentous mycelium network.

The process that is considered a cultivation or aerobic fermentation process, may take place in a stirred-tank bioreactor, airlift reactor or bubble column reactor, where the liquid medium is agitated by aeration and/or stirring. The process is advantageous compared to a so-called solid-state fermentation process in that the produced fungal biomass is essentially free of any leftover substrate particles, which would otherwise prevent obtaining the improved texture as described in the present invention. However, solid state fermentation may also be used for producing a fungal biomass, provided that the leftover substrate particles are separated from the fungal biomass. This may e.g. be effected by washing or scraping the fungal biomass from the solid substrate. Furthermore, the skilled person knowns different aerobic fermentation processes that may be used for producing fungal biomass, such as batch, fed-batch, repeated fed-batch and/or continuous mode.

Step c) may further comprise adding food additive as defined elsewhere herein, to the fungal biomass obtained and mixing the fungal biomass and the food additive, thereby integrating the food additive into the filamentous mycelium network. The food additive may be added in amount of from 0.05% by weight or more as disclosed elsewhere herein.

The present document is also directed to a dry food product comprising fungal biomass obtained or obtainable by a method as defined herein. The dry food product comprising fungal biomass may be a dry fungal biomass product as defined herein.

### BRIEF DESCRIPTION OF THE DRAWINGS

FIG. 1 shows a graph relating to the growth of fungi spores into fungi filaments, the variation in pH and the observed morphology of the mycelium in suspension.

FIG. 2. shows a graph relating to the growth of fungi spores into fungi filaments with varying concentrations of yeast extract in different media.

FIG. 3 shows images obtained through stereomicroscopy indicating alignment of fungal mycelium according to a lamellar structure and comparing samples with and without embedded food additives.

FIG. 4 shows the water content of different samples over time during the process of chilled vacuum dehydration at different sample temperatures.

FIG. 5 shows the values of water absorption capacity (WAC) over time for fungal mycelium biomass dehydrated using different methods.

FIG. 6 shows the values of water absorption capacity (WAC) after rehydration for 30 min of samples dehydrated though chilled vacuum dehydration with the samples at different temperatures, compared to the same samples freeze dry.

FIG. 7 shows texture analysis results from a knife blade and guillotine test measuring firmness and toughness values for fresh biomass samples compared to rehydrated samples from freeze drying and chilled vacuum dehydration.

FIG. 8 shows the differences in the color profile according to CIELAB scale of fungal mycelium biomass samples dehydrated with different drying methods.

FIG. 9 shows measured energy consumption of freeze drying and chilled vacuum dehydration of the same samples of fungal mycelium biomass.

FIG. 10 shows results from an energy consumption model comparing the process of freeze drying and chilled vacuum dehydration of fungal mycelium biomass, where the data is shown as a ratio between the energy consumption of the two processes.

FIG. 11 shows the WAC of rehydrated fungal mycelium biomass, chicken and HME soy protein dehydrated through freeze drying and chilled vacuum dehydration.

FIG. 12 shows the water content during a rehydration process of different dry foods, including dehydrated fungal mycelium biomass.

FIG. 13 shows the water content of rehydrated dry fungal mycelium biomass with different embedded food additives.

FIG. 14 shows the water absorption capacity of rehydrated dry fungal mycelium biomass with different embedded food additives.

FIG. 15 shows the correlation between water activity and water content of dry fungal mycelium biomass.

FIG. 16 shows an illustration of how the fungal mycelium is arranged in a fungal mycelium network lamellar structure. The “threads” in the figure illustrate fungal filaments (hyphae).

### DETAILED DESCRIPTION

The term fungal biomass refers to a biomass comprising or consisting of intact and/or lysed fungal cells. Typically, most fungal cells are intact in the fungal biomass. The terms fungal biomass and fungi biomass may be used interchangeably herein.

The term mycelium network refers to a continuous structure of fungal cells or hyphae that form a solid 3-dimensional structure.

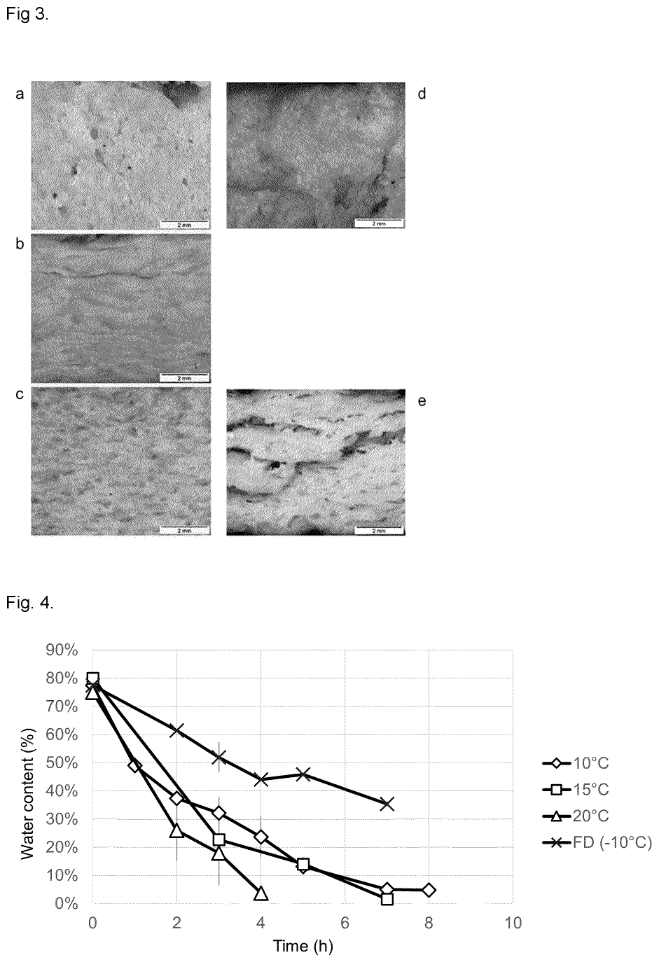

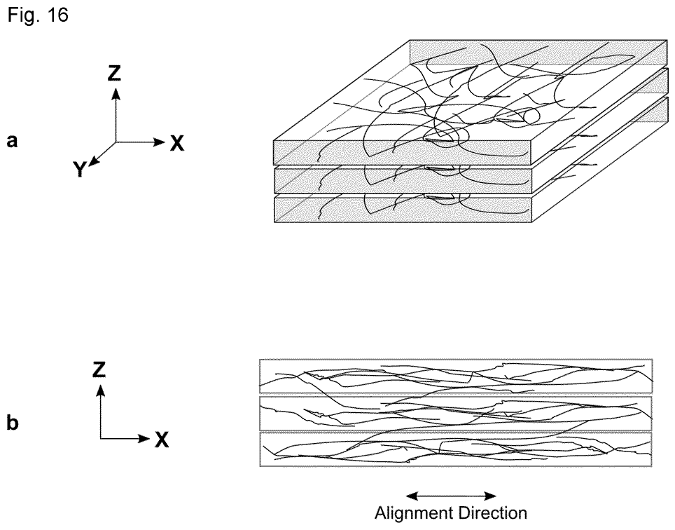

The term lamellar structure refers to a macrostructure formed by parallelly arranged planes of fungal filaments (see e.g. FIGS. 3 and 16). Within each plane, the fungi form a filamentous mycelium network wherein on a first axis, the fungal mycelium is arranged in parallel with the plane, while in the 2 other axes, the hyphae of the fungal mycelium are randomly distributed between them.

Chilled vacuum dehydration refers to a technique where a product is chilled at a temperature between 0° C.-17° C. and the water content is reduced using vacuum between 4 mbar and 50 mbar.

In the context of the present document the terms “dry food product”, “dry food product comprising fungal biomass”, “dry fungal biomass food product”, “dried food product”, “dried fungal biomass food product” and the like may be used interchangeably.

In the context of the present document the terms “consumer product”, “consumer end product” “final food product” and the like may be used interchangeably.

In the context of the present document the terms “wt %”, “% by weight” and the like may be used interchangeably.

In order to obtain a rehydratable food product based on fungal mycelium biomass, the process needs to encompass the following general steps: Production of the fungal mycelium biomass using a filamentous fungi in a liquid bioreactor culture; harvesting and processing the fungal mycelium biomass to achieve a meat-like texture and mouthfeel, as well as a neutral taste and safety of the food product; and dehydration of the fungi-based product using a technique that allows for said structure and texture to be retained upon rehydration.

Production of the fungal mycelium biomass entails the use of a species of filamentous fungi that can form mycelial structures in liquid fermentation conditions such as the ones belonging to the Zygomycota and Ascomycota phylum (excluding yeasts). Some exemplary fungi species that may be used to generate fungal mycelium biomass are as fungi of the genera Rhizopus, Neurospora, Aspergillus, Trichoderma, Pleurotus, Ganoderma, Inonotus, Cordyceps, Ustilago, Tuber, Fusarium, Pennicillium, Xylaria, Trametes, or any combination thereof. Examples of food safe filamentous fungal species include, but are not limited to, Aspergillus oryzae, Rhizopus oryzae, Rhizopus oligosporus, Rhizopus microsporus, Fusarium graminareum, Cordyceps militans, Cordyceps sinensis, Tuber melanosporum, Tuber magnatum, Pennicillium camemberti, Neurospora intermedia, Neurospora sitophila, Xylaria hypoxion or any combination thereof. For example, fungi of the genus Rhizopus may be used.

Any method commonly used for growing fungi may be used to produce the fungal biomass used in the present document. The fungal biomass may e.g. be obtained by growing fungi by liquid or solid fermentation. For example, the fungi may be grown under aerobic submerged fermentation conditions in a closed fermentation vessel with a liquid substrate medium with stirring.

The fungal cells may be inoculated either from plates or from a spore suspension into a pre-culture, which can be a flask or liquid bioreactor up to 30 L working volume. The culture media may advantageously contain a carbon source, nitrogen source, phosphates and sulphates, and a trace metal solution to enable growth of the fungi and obtaining of the biomass. The preculture volume is then e.g. used to inoculate a production bioreactor, or a subsequent seed bioreactor with a volume 10-50 times larger than the preculture volume, with a culture media using the same pre-requirements as the preculture media. The bioreactor conditions may be kept at a pH between 4.0 and 6.0, with an aeration of at least 0.1 vvm and stirred using propeller blades. The fungi are preferably grown in a mycelial state, as opposed to pellet-like structures, even if it is possible to also use a pellet-like structure of the fungi. The growth can be done in a batch mode, in which fungi are harvested from the production tank after a 24 h process or until less than 5% of the remaining carbon source is present, or as a continuous process, in which biomass is removed at a constant rate that matches growth and nutrient feed rate, or a semi-batch mode where biomass is partially harvested from one or several reactor and then such reactor(s) are filled with new media to continue fungal growth.

The fungal mycelium biomass is then harvested by separating fungal mycelium biomass from the liquid using any filtration or sieving mechanisms, such as sieve, decanter, centrifuge or filter system. Alternatively, this separation may take place after the heat treatment step.

The biomass is preferably heat treated, such as at a temperature of 50° C. and 95° C. before the dewatering step. For example, such heat treating may be performed by submerging the fungal biomass in a water bath for a temperature between 50° C. and 95° C., e.g. between 60 and 80° C., for a time between 1 min and 30 min, to degrade RNA and/or deactivate fungal cells, ensuring product safety and/or prolonging the final product shelf life.

Optionally, the fungal biomass can be washed. Such washing is typically performed before heat treatment or after the step of separating the fungal biomass from the cultivation medium. By washing the fungal biomass certain constituents of the fungal biomass may be removed, such as salts. Washing may also be applied to adjust the pH value of the fungal biomass. Hence, a washed biomass may have improved properties relating to edibility. For example, the fungal biomass may be washed with tap water, distilled water or water containing 0-5% of salt (NaCl), e.g. at a temperature of 6-15° C., for e.g. 3-60 min.

The biomass is then dewatered to a water content between 80-98%, cooled down and preferably kept at 1-17° C., such as 1-10° C. It is preferred that the fungal biomass is dewatered such that the filamentous mycelium network is substantially oriented in planes, for example such that 50% or more, such as 70% or more, or 80% or more of the filamentous mycelium network are aligned substantially in planes extending in a first direction, thus generating a lamellar structure (i.e. single planes of mycelium network placed on top of each other). Suitable devices for generating a lamellar structure may be selected from hydropress, belt press and/or centrifugation, such as batch-mode centrifugation device. The dewatering step may be preceded by a step of separating the fungal biomass from the liquid cultivation media or the washing liquid, such as by filtration, optionally such that the biomass has a water content within the range of from 80% to 98%.

Food additives may then be integrated into the fungal mycelium biomass, preferably before the dewatering step, by mixing said additives into the fungal mycelium biomass and stirring the mix so that said ingredient get embedded in-between the fungal fibres. The product comprising or consisting of either pure fungal mycelium biomass (i.e. fungal biomass) and optionally water or fungal mycelium biomass (i.e. fungal biomass) and food additives and optionally water can then be frozen.

Alternatively, the product comprising or consisting of either pure fungal mycelium biomass and optionally water or fungal mycelium biomass and food additives and optionally water can be subjected to a dewatering step using preferably a pressing mechanism, in which the product is pressed along a single direction until the water content is between 50-80 wt %, such as 50-75 wt %. The pressing can be done using any device that creates a unidirectional pressing with mechanical force such as an hydropress, and alternatively devices that exert this force through pneumatics, hydraulics or mechanical wheels. Alternatively, the fungal mycelium biomass (i.e. fungal biomass) or formulated product can be dewatered using alternative processes such as centrifugation, decanting or filter pressing. Other methods such as extrusion may promote some fiber alignment, however no lamellar structure is generated by such methods. Furthermore, other methods such as screw press, cheese press and filter press do not allow fiber expansion and hence alignment. The final product is may then be cooled down to a temperature between 0° C. and 17° C.

The chilled product at a temperature between 0° C. and 17° C., preferably between 0° C. and 10° C., may then be placed in a dehydration device capable of creating a vacuum between 0.001 to 50 mbar, such as from 1 to 50 mbar, such as from 2 mbar to 50 mbar, such as from 1 to 15 mbar, such as from 4 to 50 mbar, preferably between 4 mbar and 6 mbar. The product is subjected to vacuum until its water content is below 10% by weight or lower, preferably 8% by weight or lower.

The dehydration device is comprised of a vacuum chamber where the product is placed, able to remain intact at a pressure between 2 mbar and 50 mbar, such as 4-50 mbar. This chamber may be able to supply heat by the use of heated shelves or through radiation such as the use of infrared light. The chamber may also contain sensors for pressure, temperature of the product and temperature of the shelves. The chamber is connected to a vacuum pump or vacuum-generating device to create the desired vacuum. The chamber may also be connected to a condenser kept at temperatures between −40° C. and −90° C. where evaporated water is able to reform as solid under vacuum.

Alternatively, the product can be dehydrated in a freeze dryer, where it will be placed frozen between −5° C. and −35° C., such as −5° C. and −25° C., and subjected to vacuum between 0.001 and 6 mbar or between 0.1 and 6.1 mbar for a period between 24 h and 72 h.

The product may alternatively be a freeze-dried product having a water content within the range of from 0 to 10 wt %, optionally 10 wt % or lower or 8 wt % or lower. Optionally, within the range of from 0 to 5 wt % or 0 to 2.5 wt %. The final product is then after dewatering frozen to a temperature within the range of from −5° C. to −25° C. The product can be sized in a wide range of values before or after dehydration. It can be sized in small bits ranging from 1 mm to 12 mm for use as rehydratable mince replacements, or to be used as a TVP replacement in commercial and industrial formulation of vegan and vegetarian products requiring texturized vegetable protein. The product can also be used in larger sizes as replacements for dehydrated meat or plant-based products that are used in meals as bites, chunks or similar. Alternatively, as shown in some examples below, the product can be shaped and mechanically formulated into different forms such as but not limited to powder, flakes or granules.

The resulting dry food product is a protein-rich product with a protein content between 50% and 60% on dry weight and which may be used as a rehydratable food product when in contact with water or broth to yield a final consumer product or an ingredient to be used in further preparation and manufacturing.

Due to its hygroscopicity, the product would also preferably be stored and transported in water-impermeable packaging so that contact with water is avoided and stored in a non-humid atmosphere. These parameters prolong the product's shelf life.

### EXAMPLES

### Example 1. Production of Fungi Mycelia Biomass Through Liquid Fermentation

### Pre-Culture Preparation

A fungal spore suspension of a filamentous fungi species (Rhizopus oligosporus) that can form mycelial structures in liquid fermentation conditions and able to sporulate was prepared by flooding a PDA plate culture with 10-20 mL of sterile water and spores scraped off the surface with a disposable, sterile spreader. Spores were counted in a hemocytometer under a light microscope, and used directly as inoculum for liquid cultivations. Fungi cultures were cultivated in Erlenmeyer flasks (volumes 100-2000 mL) with or without baffles, filled with liquid growth medium to a maximum of 20% of the total flask volume. 1 mL of spore suspension (10 \hat{ }7 spores/mL) per 100 mL of growth media comprised of 20 g/L glucose, 5 g/L ammonium sulphate, 7 g/L of potassium phosphate and 1 ml/L of a trace mineral solution was added to each flask, followed by incubation at 30-35° C. for 18-24 h under shaking (100-150 rpm).

### Calculation of Dry Matter and Water Content

Water content in fungi samples and later any food samples was calculated by weighting three sample replicates before and after an oven drying step and comparing the values. The oven drying step was carried in a convection oven where samples were dried at 105° C. overnight (10-16 h). The measurement was stopped when the weight change is less than 1 mg during a 90 seconds time frame.

### Evaluation of Pre-Cultures

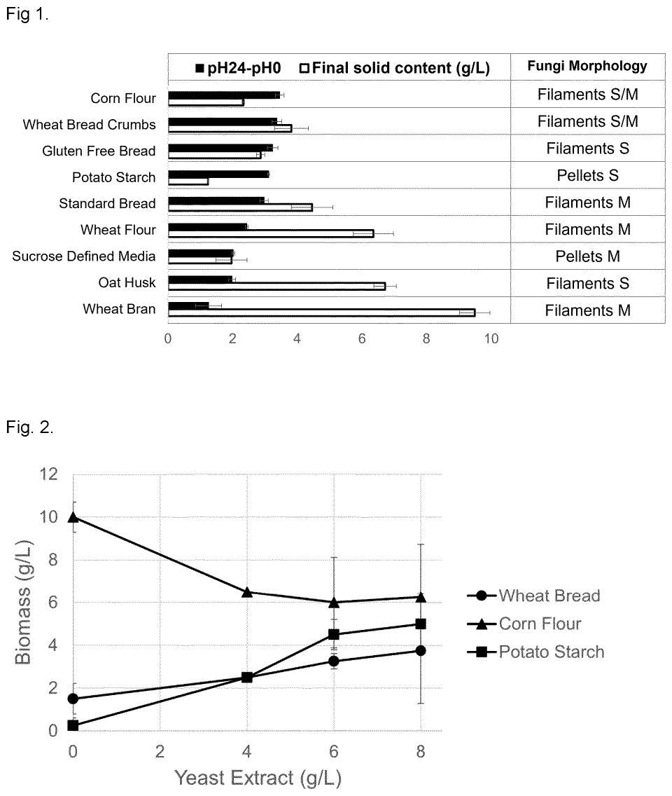

Different growth media were evaluated for biomass growth and morphology changes. Defined growth media contained either glucose or sucrose as the carbon source at 20 g/L, ammonia sulphate, potassium, magnesium and calcium in the form of phosphates, sulphates or chlorides. Germination of fungal spores into fungal mycelium in liquid media was compared between the defined growth media and other complex media. Complex media was produced by suspending 20 g/L of the ingredient. Ingredients tested included corn flour, wheat breadcrumbs, dry gluten free bread, potato starch, standard dry white bread, wheat flour, fine milled oat husks or wheat bran. Fungi cultures were performed over 24 h, initial, final, and intermediary pH values were taken, as well as the dry solid content of the culture. Growth of the fungal spores into filaments was measured by weight of the dry solid content, which includes fungi and insoluble solids, together with measurement of the decrease in pH of the culture. The mycelia macro-level morphology was also observed. All results are shown in FIG. 1. The black bars show the variation in pH between pH at 24 h and initial pH, white bars show the solid concentration at 24 h. Table on the right shows the observed morphology of the mycelium in suspension. Semi-defined rich complex media was also created using the above-mentioned potato starch, wheat bread or corn flour media and adding 4-8 g/L of yeast extract. Macro-level morphology was analyzed visually through an Erlenmeyer flask, while micro-level morphology was observed under a light microscope. Three biological replicates were compared for all observations. The addition of yeast extract was shown to be beneficial for formation of soft filamentous structures on the fungal culture when more pure carbon sources were used such as potato starch.

Nonetheless, yeast extract promoted growth of the fungi in a filamentous form in all media (Table 1) while simultaneously maintaining or increasing biomass yield (FIG. 2). Fungi from different preculture were then inoculated in a defined sucrose media for fermentation and the filamentous morphology was found to be necessary for growth of fungi into a filamentous mycelia structure during the fermentation.

TABLE 1
The table shows different morphologies of fungi in suspension
in liquid media. On the right column are specified the substrate
used in the media, while the different columns show the different
amounts of yeast extract added to such media.

| | Yeast Extract (g/L) | | | |
|---|---|---|---|---|
| Substrate | 0 | 4 | 6 | 8 |
| Wheat Bread | Open Pellets | Filaments | Filaments | Filaments |
| | M | M | L | L |
| Corn Flour | Open Pellets | Open Pellets | Open Pellets | Filaments |
| | XS | M | L | M |
| Potato Starch | Pellets | Open Pellets | Filaments | Filaments |
| | | L | S | M |

### Liquid Bioreactor Fermentation Conditions

Sterilisation of the liquid in the bioreactor was done by heating up the liquid with steam (via the bioreactor's double jacket) to 121° C. and 1 bar overpressure for 20 min. Upon sterilization, a volume of 30 L of fungi culture obtained from a 16-24 h rich media preculture was used to inoculate 300 L of media in a 400 L stirred-tank bioreactor using the media composition described previously. The pH was adjusted to 4.0-5.5 with 5M NaOH. Fermentation conditions were kept at pH 4.0 using NH3 as a base for pH titration, an air flow of 120 L/min (0.6 vvm) and a temperature of 30-35° C. were kept constant with a stirring of 200 rpm. The fermentation process was carried for 24 h and biomass was harvested after this period. 50 L from this culture was used to inoculate a volume of 500 L in a 600 L bioreactor and the process was repeated for an additional 24h.

### Determination of Protein Content

Protein content was determined on dry fungal mycelium biomass that has been freeze dried. Protein has been determined on the dry biomass using the Dumas combustion method (FlashEA 1112 Element Analyzer, Thermo Finningan, US) where nitrogen content was determined and converted to protein content with factor 6.25. Results from protein content analysis showed values between 54% and 60% of protein per dry weight among different fungal batches.

### Example 2. Enhancement of Fungal Mycelium Biomass Texture and Creating Fiber Alignment

### Texture Enhancement

Fungal mycelium biomass (Rhizopus oligosporus) was harvested from a 300 L bioreactor and subjected to a heat treatment procedure by incubating in water or culture media for 10-20 min at 65-72° C. After, it was concentrated by flowing medium with biomass through large sieve-like filters. Water content was controlled to retain more than 80% water content and to the point in which its behaviour was one of a viscous liquid and not a solid. The biomass was mixed at a constant stirring rate for 5 minutes in the presence of either rice starch, potato starch, corn starch as well as other modified starches, potato fibers, bamboo fibers, pea fibers, oat fibers, canola oil, pea protein, soy protein, and hydrocolloids such as methylcellulose, carrageenan, and alginate in an amount of 0.25%, 0.5%, 2%, 5% and 10% relative to the final wet product weight.

### Dewatering and Pressing

Samples of biomass with embedded additives were pressed in a hydropress system using a water pressure of 2.0-2.5 bar, in which force is applied in a single plane to simultaneously dewater the samples and promote fiber alignment. Water content in the samples was decreased from 80-99.9% in which the biomass has a liquid behaviour from low to very-high viscosity, to 63-80% where the biomass is in a wet solid form.

### Stereomicroscopy Analysis of Pressed Biomass Comprising Fibers Integrated in the Filamentous Mycelium Network

Fungal mycelium biomass was produced and pressed as described above with either no additives added during production, or by adding food additive such that the fungal mycelium biomass contained 4% fibres and 2% canola oil integrated in the filamentous mycelium network according to the present disclosure. Samples were generated by cross-cutting surfaces with a razor blade while frozen (“Cross-Cut”) or tearing by hand after defrosted (“Tearing”). These samples were then examined with Zeiss SteREO Discovery.V8 stereomicroscope equipped with Achromat S 0.5×objective (Carl Zeiss Microlmaging GmbH, Gttingen, Germany) and imaged using an Olympus DP-25 single chip colour CCD camera (Olympus Life Science Europa GmbH, Hamburg, Germany) and the Cell\hat{ }P imaging software (Olympus).

The obtained images in FIG. 3 show that the fungal mycelial network is substantially aligned forming a lamellar structure. FIG. 3a represents a cross-cut of fungal biomass without pressing, and 3d is from tearing the biomass. 3b shows cross-cut of pressed biomass, 3c cross cut of pressed biomass with additives, and 3e tearing of pressed biomass with additives. Where additives are present, these are seen embedded in the mycelial network. When tearing or simulating a bite, the sample with embedded fibre and oil additives was shown to separate according to its lamellar structure and reveal aligned mycelial fibres.

### Example 3. Dehydration of Fungal Mycelium Biomass

### Dehydrating Through Conventional Drying Methods

Fungal mycelium biomass obtained from Example 1 was cut in cubes of 1 cm and dehydrated using conventional hot air drying through wither a convection oven or a conduction oven. The fungal mycelium biomass was dried at 50° C. or 70° C. for 6 h or overnight (until there was no significant change in sample weight). Samples dried through hot air drying resulted in a dark-coloured, compact and extremely hard mass.

The fungal mycelium biomass from example 1 was also freeze dried. For this, the biomass was cut in cubes of 1 cm and frozen for 24 h at −20° C. After frozen, samples were placed in an Alpha 1-4 LSCplus freeze dryer set to shelf temperature of −10° C., vacuum between 1 and 3 mbar using a rotary vacuum pump, and condenser temperature at −86° C. The product temperature was monitored, and the drying was deemed complete when the product did not show a cooling from ongoing sublimation. The time to a dry product averaged at 64h. Products from freeze dry showed a bright white colour similar to the fresh product, with an intact structure similar to the original product.

### Dehydrating Through Chilled Vacuum Dehydration

The fungal mycelium biomass from both Example 1 and 2 was subjected to a customized vacuum process at low temperature. The samples were cut in 1 cm cubes and chilled in a fridge to a stable temperature of 10° C., 15° C. or 20° C. The samples were then placed in a vacuum chamber with shelves regulated to be kept at 10° C., 15° C. or 20° C. respectively. The samples were spread among the shelves so that all cubes would be in contact with the regulated surface. The chamber was also connected to a condenser with a temperature between −50° C. and −86° C. The chamber was subjected to a vacuum pressure of 4 mbar. Samples were collected every hour and water content was calculated for these samples as explained in Example 1.

The results shown in FIG. 4 show that an increased temperature creates a faster dehydration process, being completed at 7 h for the processes at 10° C. and 15° C., and 4 h for the 20° C. process. Samples from this chilled vacuum dehydration process (CVD) at 10° C. and 15° C. had a similar visual appearance as samples from freeze drying, but samples dried at 20° C. showed high variance in their appearance, and some would appear with a brown, compact look.

### Example 4. Rehydration of Dry Fungal Mycelium Biomass and Evaluation of the Product from Different Dehydration Methods

Water Absorption Capacity Measurements of Fungal Mycelium Biomass from Different Drying Methods

Samples obtained from freeze drying, hot air drying (convection and conduction oven) and chilled vacuum dehydration at 10° C. in Example 3 were used to evaluate the capacity to rehydrate when in contact with water. The weight of the samples was taken before and after dehydration, then samples were submerged in excess water at a room temperature of approximately 23° C., with their wet weight measured at 1, 3, 5, 10, 15, 30, 60, 90 and 120 minutes. The water absorption capacity (WAC) was calculated as WAC=(Wr−Wd)/(Wo−Wd), where Wo is the weight of initial sample, Wd the weight of dry sample and Wr the weight of rehydrated sample.

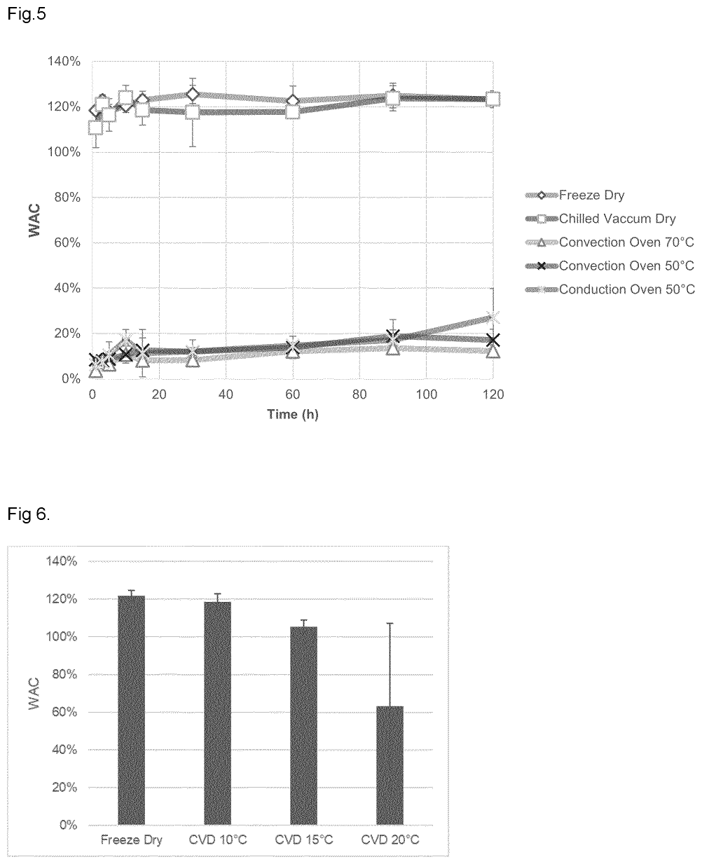

The results shown in FIG. 5 show that both freeze drying and chilled vacuum dehydration results in highly rehydratable samples with a WAC of about 120%, while hot air drying methods result in a n on-rehydratable sample with a WAC below 20%.

Regarding different temperature of chilled vacuum dehydration, the WAC of samples rehydrated for 30 min was measured between freeze dried and chilled vacuum dried samples. As shown in FIG. 6, the WAC of samples dehydrated at 10° C. was not significantly different from the freeze dried samples, while the samples dehydrated at 15° C. had a WAC reduced from 120% to about 105%. Samples dehydrated at 20° C. had a large variability of WAC values, with an average of 60%.

### Texture Analysis of Rehydrated Biomass

Samples were prepared as explained in Example 3 and 4. Rehydrated samples from freeze drying and chilled vacuum dehydration at 10° C. were evaluated regarding its texture profile of biting, for which texture analysis using a Knife Blade method was used. Samples of solid fungal mycelium biomass were prepared as a cuboid shape of 20 mm×10 mm×5 mm (length×width×height) for texture analysis. Texture analysis was carried using a Stable Microsystems TA.TX Plus-C equipped with a Knife Blade (70 mm width×3 mm thick, 45° chisel end) and guillotine block. The sample was placed in the centre of the guillotine block and cut with the knife blade starting at a position of 20 mm and a descending speed of 2 mm/s for 30 mm. A curve plot was obtained showing measured Force×Time, and the parameters of Firmness was defined as the maximum Force value of the curve in g, while Toughness was defined as the total area below the curve in g-s.

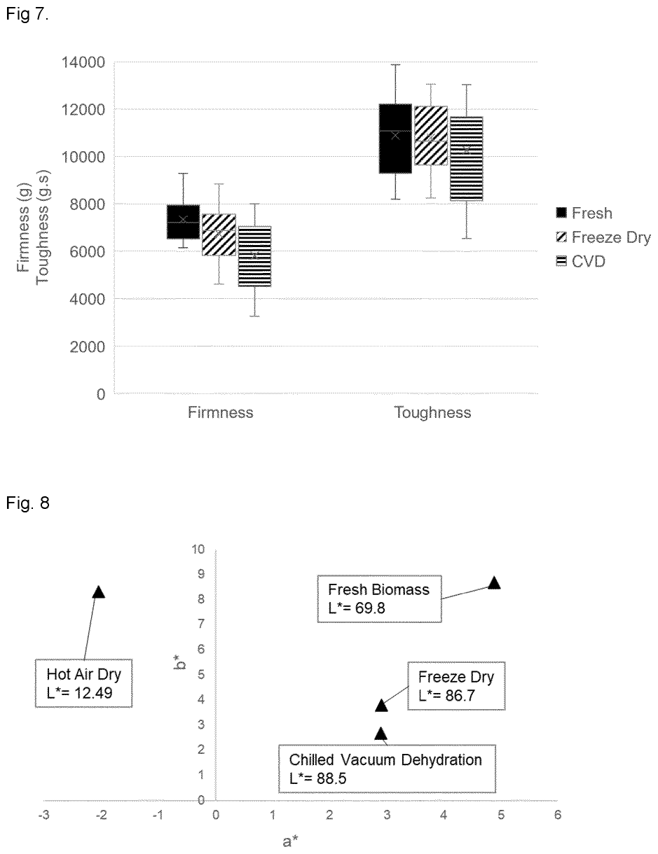

The results from the testing are illustrated in FIG. 7, where the fresh fungal mycelium biomass (frozen and thawed, non-dehydrated) was compared with the same biomass either freeze dried or chilled vacuum dried at 10° C. The results show a similar profile of Firmness and toughness, with no statistical differences between them. Chilled vacuum dehydration seems to provide a less firm and tough texture, but only by a marginal difference. Samples from hot air drying were also analyzed but its values would overshoot the load cell, indicating extremely high firmness and toughness values, unpleasant for any food product, that could not be measured with the current setup.

Color comparison of fungal mycelium biomass from different dehydration methods Samples of fungal mycelium biomass were obtained as explained in Example 1 and dehydrated using convection oven drying at 50° C., freeze drying or chilled vacuum dehydration at 10° C. as explained in Example 3. A camera was setup in a lightbox with a standardized distance from camera to sample, lightning, position and camera manual settings. Photos of the samples of fungal mycelium biomass both fresh and dehydrated were taken and analyzed using ImageJ. In ImageJ, an homogenous area was defined and the average colour values were taken and translated into the CIELAB scale. Results for a*, b* and L* are shown in FIG. 8, where it shows a high similarity in colour between freeze dry and chilled vacuum dry samples, which exhibit a light white/beige colour. These are highly different from oven dry samples, which have a brown dark colour.

### Example 5. Production of Dry Fungal Mycelium Biomass from Different Fungal Morphologies

Fungal mycelium biomass with different morphologies obtained from Example 1 were obtained. One set of samples had a filamentous morphology while another set had a pellet-like morphology. Samples were dehydrated using chilled vacuum dehydration and rehydrated as explained in Example 3. Filamentous morphology samples had a water absorption of 3.6 (±0.13) g water/g dry matter, while Pellet morphology samples 4.2 (±0.18) g water/g dry matter, showing that morphology has a small impact in water absorption capacity of dry fungal mycelium biomass, but not enough to be relevant in practice.

### Example 6. Use of Chilled Vacuum Dehydration for Cost Reduction of High Quality Dry Fungal Mycelium Biomass

### Experimental Comparison of Energy Consumption of Freeze-Drying Vs Chilled Vacuum Dehydration Process

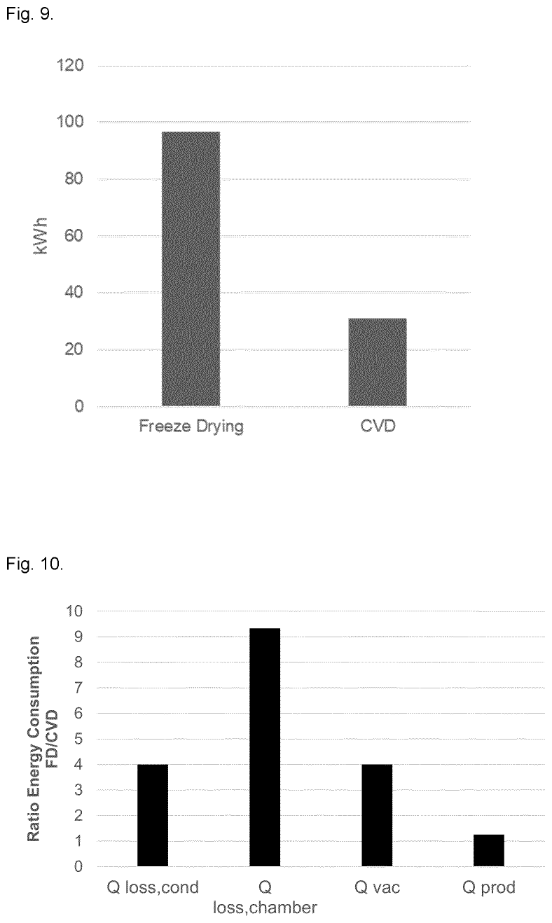

To quantify energy consumption, an electricity meter was used to measure the consumption of energy during the freeze drying and chilled vacuum dehydration processes. The chilled vacuum dehydration operated with a shelf and sample temperature of 10° C., condenser temperature of −86° C., vacuum pressure of 4 mbar, and the sample dehydration was completed after 7 h. For the freeze drying process, a shelf and sample temperature of −10° C. was used, condenser temperature of −86° C., vacuum pressure of 1 mbar, and the sample dehydration was completed after 64 h. The results shown in FIG. 9 show a 68% decrease in electricity consumption by using the chilled vacuum dehydration process.

### Model Analysis of Energy Consumption of Freeze Drying Vs Chilled Vacuum Dehydration Process

A mathematical model was also built to simulate power consumption of freeze drying versus chilled vacuum dehydration processes at larger scale. For this, the model did not account for the energy require to freeze or chill the product, and assumes only one phase of freeze drying. In FIG. 10 it is shown the values output from the model concerning the ratio of energy consumed between freeze drying and chilled vacuum dehydration. The parameters are describes and calculated by the following:
Q_{prod}: the consumption required to vaporize/sublimate the product
Q_{vac}: the energy needed by the vacuum pump to lower and maintain the pressure.
Qₗₒₛₛ: the energy needed to compensate the loss of heat

$${Q}_{p r o d} = {m}_{p r o d} {x}_{w} {λ}_{s u b / v a p} ϕ \qquad ( 1 )$$

Where m_{prod} is the mass of product; x_{w} is the water content; λ_{sub/vap} is the latent heat of sublimation or vaporization and ϕ is the part of water remove of the product. It is assumed that the product is already at the temperature and the temperature is constant.

$${Q}_{v a c} = {P}_{N} * {t}_{D} \qquad ( 2 )$$

Where P_{N} is the power of the pump; t_{D} is the time of drying. It is assumed that the time and the difference of power to low the pressure is insignificant in view of the time and the small volume of dryer.

$${Q}_{loss} = {Q}_{loss , chamber} + {Q}_{loss , cond} \qquad ( 3 )$$

Where Q_{loss,chamber} is the energy loss of the chamber in the atmosphere and Q_{loss,condenser} is the energy loss of the condenser in the atmosphere.

The chambers are considered as two cylinders with on the bottom the condenser and on the top the chamber.

$${Q}_{loss , chamber} = {U}_{chamber} {A}_{c h a m b e r} ( {T}_{a m b i a n {t}^{-}} {T}_{c h a m b e r} ) {t}_{D} \qquad ( 4 )$$

$${Q}_{loss , cond} = {U}_{c o n d} {A}_{c o n d} ( {T}_{a m b iant} - {T}_{c h a m b e r} ) {t}_{D} \qquad ( 5 )$$

U are the global heat transfers coefficients and A are the surfaces of the chamber and condenser. The following values are assumed: x_{W}=0.75 and ϕ=0.98; Chilled vacuum dehydration: t_{D}=16 h, T_{A}=25° C. and T_{D}=10° C., Freeze Drying: t_{D}=64 h, T_{A}=25° C., T_{D}=−10° C.

### Example 7. Using Chilled Vacuum Dehydration for Production of Different Dry Protein Products

### Use of Chilled Vacuum Dehydration for Varied Protein Food Products

Cubes of fungal mycelium biomass were obtained as described in Example 2, chicken breast was boiled and cubes of 1 cm, high moisture extruded (HME) soy protein was obtained from commercial sources. For chilled vacuum dehydration (CVD), all products were processed as explained in Example 3 using 10° C. product and shelf temperature. Samples were dehydrated for about 12 h. Samples were submerged in water for 3 minutes and water content measured to calculate water absorption capacity (WAC), and the results are shown in FIG. 11. Fungal mycelium biomass shown to have higher WAC than chicken or HME soy protein. Samples obtained through CVD were equivalent as samples from freeze drying for fungal mycelium biomass samples, and statistically close for chicken. A bigger difference was observed for HME soy protein.

### Example 8. Use of Dry Fungal Mycelium Biomass as a Replacement to Dry Protein Products

### Rehydration of Freeze Dry and Chilled Vacuum Dehydrated Fungal Mycelium Biomass Compared to Other Rehydratable Food Products

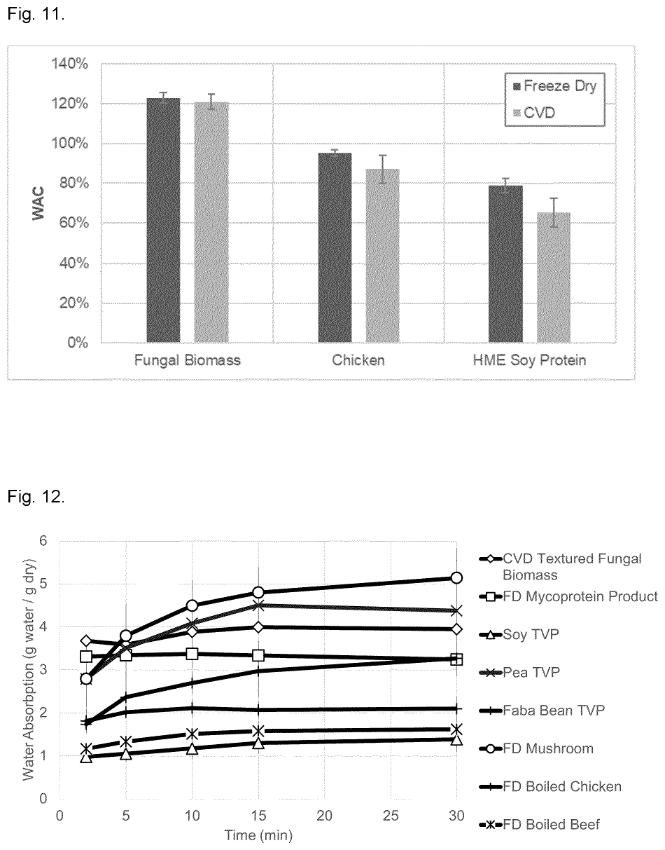

Dry fungal mycelium biomass was obtained by Freeze Drying or Chilled Vacuum Dehydration as described in Example 3. Samples of white mushrooms, boiled chicken, boiled beef and a chicken-like mycoprotein-based commercial product were also frozen and freeze-dried using the same method as explain in Example 3. The dehydrated samples were then compared between each other and to other rehydratable protein products such as Pea textured vegetable protein (TVP), Soy TVP and Faba Bean TVP. The dry products were placed in contact with excess water, and water content was calculated as described in Example 1. At selected times of 2, 5, 10, 15 and 30 min the samples were removed and water content measured. The results of water absorbed over time are shown in FIG. 12, showing that CVD Fungal mycelium biomass has the quickest rehydration rate between 0-2 min and a water holding capacity only surpassed by Pea TVP and freeze dry mushrooms.

### Example 9. Inclusion of Functional and Flavour Ingredients in Dry Fungal Mycelium Biomass

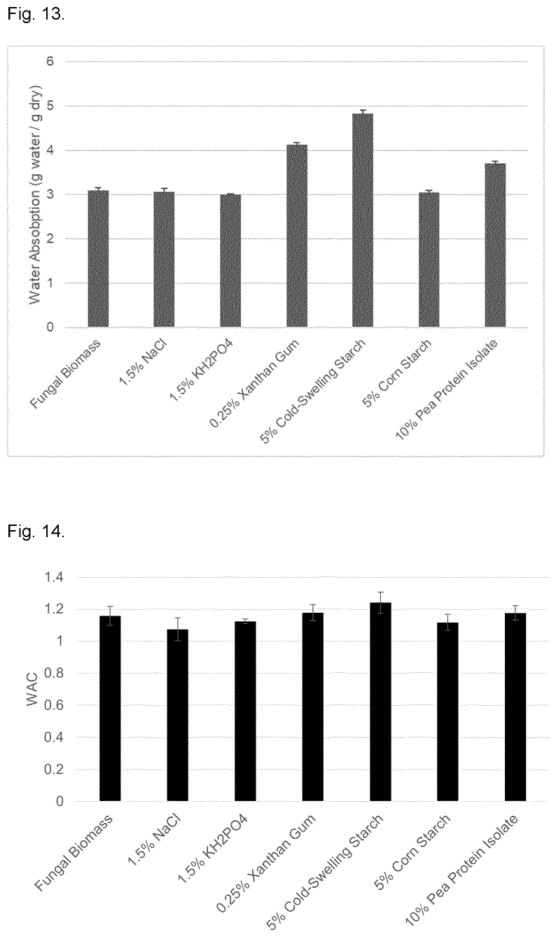

Fungal mycelium biomass was processed like in Example 2, in which biomass was mixed with either 1.5% NaCl, 1.5% KH2PO4, 0.25% xanthan gum, 5% modified cold-swelling starch, 5% corn starch, or 10% pea protein isolate. The biomass was then pressed and cut as explained in the example. The resulting product was then dehydrated using chilled vacuum dehydration as explained in Example 3. Samples were rehydrated and the water absorbed was calculated from the resulting weight of the samples, shown in FIG. 13. The WAC was also calculated as shown in FIG. 14. We could see that the inclusion of food additives can increase the amount of water held by the material if such additive is also known to bind water such as the experimented hydrocolloids. This however, does not disrupt the WAC properties of the material.

Different flavours and spices were also included in the processed biomass. It was observed that both texturizing additives but also flavouring and spices can remain in the mycelium structure and be recognized sensorially after dehydration and rehydration of the material.

### Example 10. Evaluation of Water Activity as Shelf Life Parameter of Dry Fungal Mycelium Biomass

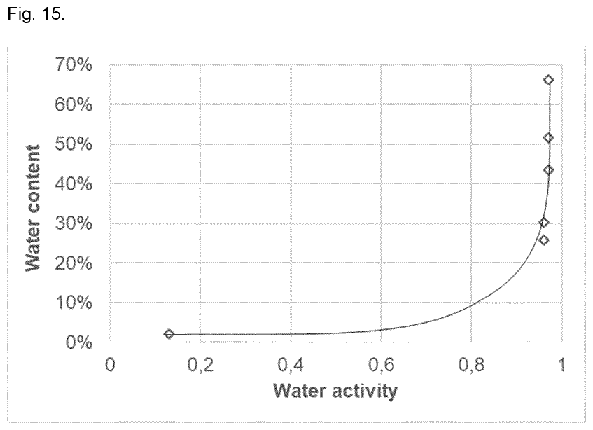

Fungal mycelium biomass was produced and dehydrated as explained in Example 3. Samples were taken on selected timepoints and dry matter content was analyzed. In parallel, samples from the same timepoints were evaluated for water activity by an external laboratory. The results in FIG. 15 show a water activity of 0.8 at 10% water content, and lower than 0.6 at a water content lower than 10%. This indicates that the dry fungi mycelial biomass will be safe from most microbial growth (pathogenic bacteria, yeasts and most moulds) at 10% water content, and safe from almost all microbial growth below this value. At 18% water content, a water activity of 0.89 was observed, indicating already an inhibitory effect to bacterial growth at this level of water content.

### Example 11. Use of Dry Fungal Mycelium Biomass as an Intermediate Ingredient in Formulation of Consumer Products

Fungal mycelium biomass was produced as in Example 3, in which the biomass with embedded additives was cut into bits between 4 and 12 mm before dehydration through chilled vacuum dehydration.

The particles were then hydrated with water at a ratio of either 1:1 or 1:2 (1 part dry biomass to 2 parts water). Instead of water, broth containing flavours was also used. The rehydrated material was mixed 1:1 with a formed patty mix consisting of 5% methylcellulose, 10% pea protein isolate, 5% starch, 28% canola oil and 54% water. A patty was formed with this mix.

The patty sensorially behaved similar to the same formulation if Pea TVP was used instead of dehydrated fungal mycelium biomass.

### Example 12. Use of Dry Fungal Mycelium Biomass as a Non-Animal Protein Replacement in or as a Final Consumer Product

Fungal mycelium biomass was produced as in Example 3, in which the biomass with embedded additives, flavours and spices was cut into bits between 1 and 10 cm long, with 1.5 cm thickness, before dehydration through chilled vacuum dehydration. On a parallel experiment, the same pieces were produced, but the resulting pieces were fried in a pan or baked until a cooked brown surface was achieved, the pieces were cut after that step with the appearance of pieces of grilled meat.

The dehydrated pieces were included in a dehydrated instant meal composed of noodles, dehydrated vegetable pieces, and seasonings as powder. The dry meal containing the dehydrated biomass pieces was rehydrated using water either at room temperature or at 80-95° C. The pieces rehydrated to completion together with the rest of the meal in 10 minutes, uptaking the soluble powder flavours that dissolved into the water.

### Example 13 Use of Dry Fungal Mycelium Biomass for Production of a Powder of High Protein Content

Dehydrated fungal mycelium biomass obtained from chilled vacuum dehydration at 10° C., freeze drying and convective oven drying at 50° C. was grinded through a mill in a “fine” particle setting. 1 g of powder was hydrated with excess water for 5 minutes and filtered. The wet mass was weighted and the water holding capacity (WHC) was calculated as:

WHC=(weight of rehydrated biomass−weight dry biomass)/weight dry biomass

TABLE 2
Water holding capacity (WHC), oil holding capacity
(OHC) and solubility of fungal mycelium biomass
powder created from different drying methods.

| | WHC | |
|---|---|---|
| | (g water/g dry) | Stdev |
| Chilled vacuum | 6.16 | 0.57 |
| dehydration | | |
| Freeze Drying | 6.11 | 0.19 |
| Hot Air Drying | 1.22 | 0.10 |

The WHC of freeze dried and chilled vacuum dry fungal mycelium biomass powder was similar and about 5-fold higher than hot air-dry biomass powder. This represents a loss of hydrophilic capacities when fungal biomass is dried using hot air, making freeze dry and chilled vacuum dry highly more suitable to be used as powders.

### Items

1. A dry food product comprising fungi biomass, the fungi biomass comprising a filamentous mycelium network and wherein the dry food product has a water content within the range of from 0 to 18 wt. %.
2. The dry food product according to item 1, wherein the the dry food product has a water content within the range of from 0 to 10 wt. %, optionally within the range of from 0 to 5 wt. %.
3. The dry food product according to item 1 or 2, wherein 50% or more, such as 70% or more, or 80% or more of the filamentous mycelium network are aligned substantially in planes extending in a first direction, thus forming a lamellar structure.
4. The dry food product according to any one of items 1-3, wherein the dry food product has a water activity of 0.8 or lower, optionally 0.6 or lower.
5. The dry food product according to any one of the preceding items, wherein the dry food product is a controlled low-temperature vacuum dehydrated food product.
6. The dry food product according to any one of items 1 to 4, wherein the dry food product is a freeze-dried food product, such as a freeze-dried consumer end product.
7. The dry food product according to any one of the preceding items, wherein the dry food product has a water absorption capacity (WAC) within the range of from 70% to 200%, optionally within the range of from 80% to 180%, optionally within the range of from 90% to 180%, according to the water absorption capacity (WAC) test as disclosed herein.
8. The dry food product according to any one of the preceding items, wherein the dry food product has a maximum rehydration rate within the rate of from 0 to 15 minutes, preferably 0 to 3 minutes, such as 0.1 to 3 minutes, according to the rehydration rate method as described herein.
9. The dry food product according to any one of the preceding items, wherein a food additive is present in an amount of 0.05% by weight or more of the total weight of the fungi biomass and the food additive, wherein the food additive is integrated in the filamentous mycelium network.
10. The food product according to any one of the preceding items, wherein the filamentous mycelium network comprising the integrated food additive is substantially intact.
11. The food product according to any one of the preceding items, wherein the integrated food additive is selected from the group consisting of food fibers, starches, proteins, fats, oils, food flours, hydrocolloids, and gelling agents.
12. The food product according to any one of items 1 to 11, wherein the integrated food additive is selected from the group consisting of rice starch, potato starch, corn starch as well as other modified starches, potato fibers, bamboo fibers, pea fibers, oat fibers, canola oil, pea protein, soy protein, hydrocolloids such as methylcellulose, carrageenan and alginate, flavours and/or spices.
13. The food product according to any one of the preceding items, wherein the food product is a dehydrated and rehydratable mince-like intermediate or final food product.
14. The food product according to any one of the preceding items, wherein the food product is packed in a water-impermeable package.
15. The use of the food product according to item 14 for providing a rehydratable ready-to-eat product.
16. A method for manufacturing a dried fungi biomass food product, the method comprising the steps of:
   a) cultivating fungi under aerobic submerged fermentation conditions using a closed fermentation vessel with liquid substrate media while stirring to obtain a fungi biomass comprising a filamentous mycelium network;
   b) processing the fungi biomass obtained from step a) by heating to a temperature within the range of from 50 to 95° C.;
   c) separating the fungi biomass obtained from step b) from the liquid cultivation media, such as by filtration, optionally such that the biomass has a water content within the range of from 80% to 98%;
   d) dewatering, such as by pressing or centrifuging, the fungi biomass obtained from step c) to substantially orient the filamentous mycelium network in a single plane, optionally such that a fungi biomass food product is obtained having a water content within the range of from 50 to 80% by weight, as measured by weighing of the fungi biomass before and after an oven drying step;
   e) cooling the fungi biomass to a temperature within the range of from 0° C. to 17° C.;
   f) maintaining the fungi biomass in a temperature within the range from 0° C. to 17° C. and subjecting the cooled fungi biomass from step e) to a vacuum pressure within the range of from 2 mbar to 50 mbar until the water content is 10% by weight or lower, optionally 8% by weight or lower.
17. A method for manufacturing a dried fungi biomass food product, the method comprising the steps of:
   a) cultivating fungi under aerobic submerged fermentation conditions using a closed fermentation vessel with liquid substrate media while stirring to obtain a fungi biomass comprising a filamentous mycelium network;
   b) processing the fungi biomass obtained from step a) by heating to a temperature within the range of from 50 to 95° C.;
   c) separating the fungi biomass obtained from step b) from the liquid cultivation media, such as by filtration, optionally such that the biomass has a water content within the range of from 80% to 98%;
   d) dewatering, such as by pressing or centrifuging, the fungi biomass obtained from step c) to substantially orient the filamentous mycelium network in a single plane, such that a fungi biomass food product is obtained having a water content within the range of from 50 to 80% by weight, as measured by weighing of the fungi biomass before and after an oven drying step;
   e) freeze-drying the fungi biomass to a temperature within the range of from −5° C. to −35° C.;
   f) maintaining the fungi biomass in a temperature within the range from from −5° C. to −35° C. and subjecting the freeze-dried fungi biomass from step e) to a vacuum pressure within the range of from 0.001 mbar to 6 until the water content is 10% by weight or lower, optionally 8% by weight or lower, and
   g) preparing a freeze-dried fungi biomass food product.
18. The method according to item 16, wherein step e) includes cooling the fungi biomass to a temperature within the range of from 0° C. to 15° C.; and step f) maintaining the fungi biomass in a temperature within the range from 0° C. to 15° C.
19. The method according to items 16 or 18, wherein step e) includes cooling the fungi biomass to a temperature within the range of from 0° C. to 12° C.; and step f) maintaining the fungi biomass in a temperature within the range from 0° C. to 12° C.
20. The method according to any one of items 16-19, wherein step f) is performed in a vacuum chamber.
21. The method according to item 20, wherein the vacuum chamber is connected to a condenser, optionally the condenser having a temperature within the range of from −50° C. to −90° C.
22. The method according to any one of items 16 to 21, wherein step c) further comprises adding food additive to the fungi biomass obtained and mixing the fungi biomass and the food additive, thereby integrating the food additive into the filamentous mycelium network.
23. The method according to any one of items 16 to 22, wherein step d) includes dewatering the fungi biomass by pressing the fungi biomass with a force applied in a single direction, optionally with a pressure of from 1.0 to 3.0 bar.

1. A dry food product comprising fungal biomass, the fungal biomass comprising a filamentous mycelium network, wherein the dry food product has a water content within the range of from 0 to 18 wt %, and wherein 50% or more of the filamentous mycelium network are aligned substantially in planes extending in a first direction, thus forming a lamellar structure.

2. The dry food product according to claim 1, wherein the the dry food product has a water content within the range of from 0 to 10 wt %.

3. The dry food product according to claim 1, wherein the dry food product has a water activity of 0.8 or lower.

4. The dry food product according to claim 1, wherein the dry food product is a controlled low-temperature vacuum dehydrated food product.

5. The dry food product according to claim 1, wherein the dry food product is a freeze-dried food product.

6. The dry food product according to claim 1, wherein the dry food product has a water absorption capacity (WAC) within the range of from 70% to 200%.

7. The dry food product according to claim 1, wherein the dry food product has a maximum rehydration rate from 0 to 15 minutes.

8. The dry food product according to claim 1, wherein a food additive is present in an amount of 0.05% by weight or more of the total weight of the dry food product, wherein the food additive is integrated in the filamentous mycelium network, wherein the filamentous mycelium network comprising the integrated food additive is substantially intact.

9. (canceled)

10. The dry food product according to claim 8, wherein the integrated food additive is selected from one or more of the group consisting of food fibers, starches, proteins, fats, oils, food flours, hydrocolloids, and gelling agents.

11. The dry food product according to claim 8, wherein the integrated food additive is selected from one or more of the group consisting of rice starch, potato starch, corn starch as well as other modified starches, potato fibers, bamboo fibers, pea fibers, oat fibers, canola oil, pea protein, soy protein, hydrocolloids such as methylcellulose, carrageenan and alginate, flavours and spices.

12. The dry food product according to claim 1, wherein said dry food product is an instant and/or rehydratable meal product.

13. The dry food product according to claim 1, wherein said dry food product is a dehydrated and rehydratable mince-like intermediate or consumer product.

14. The dry food product according to claim 1, wherein said fungi are food-safe filamentous fungi.

15. The dry food product according to claim 14, wherein said food-safe filamentous fungi are fungi of the genera Rhizopus, Neurospora, Aspergillus, Trichoderma, Pleurotus, Ganoderma, Inonotus, Cordyceps, Ustilago, Tuber, Fusarium, Pennicillium, Xylaria, Trametes, or any combination thereof.

16. The dry food product according to claim 15, wherein said food-safe filamentous fungi are fungi of the species Aspergillus oryzae, Rhizopus oryzae, Rhizopus oligosporus, Rhizopus microsporus, Fusarium graminareum, Cordyceps militaris, Cordyceps sinensis, Tuber melanosporum, Tuber magnatum, Pennicillium camemberti, Neurospora intermedia, Neurospora sitophila, Xylaria hypoxion or any combination thereof.

17. (canceled)

18. (canceled)

19. A method for manufacturing a dried fungal biomass food product, the method comprising the steps of:
a) providing a solution or suspension comprising a fungal biomass, such as a fungal biomass comprising food-safe filamentous fungi as defined in claim 14;
b) dewatering the fungal biomass to substantially orient the fungal biomass in a filamentous mycelium network in planes thus forming a lamellar structure, said dewatering being made by applying a unidirectional pressing force to the fungal biomass; and
c) submitting the dewatered fungal biomass to chilled vacuum dehydration and/or freeze-drying, thereby obtaining a dried fungal biomass food product.

20. The method according to claim 19, wherein said fungal biomass is heat treated before or after step a).

21. The method according to claim 19, wherein said method further comprises adding a food additive to the fungal biomass before step b) and mixing the fungal biomass and the food additive, thereby integrating the food additive into the filamentous mycelium network.

22. The method according to claim 19, wherein step c) comprises the steps of:
i) cooling the fungal biomass obtained in step b) to a temperature within the range of from 0° C. to 17° C.;
ii) maintaining the fungal biomass at a temperature within the range from 0° C. to 17° C. and subjecting the cooled fungal biomass to a vacuum pressure within the range of from 0.001 to 50 mbar until the water content is 10% by weight or lower, thereby obtaining a dried fungal biomass food product.

23. The method according to claim 19, wherein step c) comprises the steps of:
i) freeze-drying the fungal biomass to a temperature within the range of from −5° C. to −35° C.;
ii) maintaining the fungal biomass in a temperature within the range from −5° C. to −35° C. and subjecting the freeze-dried fungal biomass from step e) to a vacuum pressure within the range of from 0.001 mbar to 6 mbar until the water content is 10% by weight or lower, thereby obtaining a freeze-dried fungal biomass food product.

24. The method for manufacturing a dried fungal biomass food product according to claim 19, the method comprising the steps of:
a) providing a fungal biomass by cultivating fungi under aerobic submerged fermentation conditions using a closed fermentation vessel with liquid substrate media while stirring to obtain a fungal biomass;
b) processing the fungal biomass obtained from step a), by heating to a temperature within the range of from 50 to 95° C.;
c) optionally separating the fungal biomass obtained from step b) from the liquid cultivation media, optionally such that the biomass has a water content within the range of from 80 wt % to 98 wt %, optionally step c) may be performed before step b);
d) dewatering by pressing or centrifuging, the fungal biomass obtained from step c) to substantially orient fungal biomass in a filamentous mycelium network in planes thus forming a lamellar structure;
e) cooling the fungal biomass obtained in step d) to a temperature within the range of from 0° C. to 17° C.;
f) maintaining the fungal biomass at a temperature within the range from 0° C. to 17° C. and subjecting the cooled fungal biomass from step e) to a vacuum pressure within the range of from 0.01 mbar to 50 mbar until the water content is 10% by weight or lower, thereby obtaining a dried fungal biomass food product.

25. (canceled)

26. (canceled)

27. The method for manufacturing a dried fungal biomass food product according to claim 19, the method comprising the steps of:
a) providing a fungal biomass by cultivating fungi under aerobic submerged fermentation conditions using a closed fermentation vessel with liquid substrate media while stirring to obtain a fungal biomass;
b) processing the fungal biomass obtained from step a), by heating to a temperature within the range of from 50 to 95° C.;
c) optionally separating the fungal biomass obtained from step b) from the liquid cultivation media, such that the biomass has a water content within the range of from 80 wt % to 98 wt %, optionally step c) may be performed before step b);
d) dewatering, by pressing or centrifuging, the fungal biomass obtained from step c) to substantially orient the fungal cells in a filamentous mycelium network in planes, thus forming a lamellar structure;
e) freeze-drying the fungal biomass to a temperature within the range of from −5° C. to −35° C.;
f) maintaining the fungal biomass in a temperature within the range from −5° C. to −35° C. and subjecting the freeze-dried fungal biomass from step e) to a vacuum pressure within the range of from 0.001 mbar to 6 mbar until the water content is 10% by weight or lower, thereby obtaining a dried fungal biomass food product.

28. (canceled)

29. (canceled)

30. The method according to claim 19, wherein the dewatering step includes dewatering the fungal biomass by pressing the fungal biomass with a force applied in a single direction.

31.-33. (canceled)
